(19) United States
(12) Patent Application Publication
(10) Pub. No.: US 2021/0097884 A1
(43) Pub. Date: Apr. 1, 2021

(54) WORK SUPPORT APPARATUS, WORK SUPPORT SYSTEM, AND WORK SUPPORT METHOD

(71) Applicant: HITACHI, LTD., Tokyo (JP)

(72) Inventors: Daisuke Tsutsumi, Tokyo (JP); Youichi Nonaka, Tokyo (JP); Takahiro Nakano, Tokyo (JP); Yumiko Ueno, Tokyo (JP)

(73) Assignee: HITACHI, LTD., Tokyo (JP)

(21) Appl. No.: 17/004,056

(22) Filed: Aug. 27, 2020

(30) Foreign Application Priority Data
Sep. 26, 2019 (JP) 2019-174967

(51) Int. Cl. G09B 19/00; G06Q 10/06; G06Q 50/04; G16H 40/67; G16H 50/30; G09B 5/02; G09B 5/06

(52) U.S. Cl. CPC G09B 19/003; G06Q 10/06316; G06Q 10/063114; G06Q 50/04; G06Q 10/06395; G09B 5/065; G16H 40/67; G16H 50/30; G06Q 10/06312; G09B 5/02; G06Q 10/06398

(57) ABSTRACT

A work capacity analysis is performed to improve productivity of a worker. A work support apparatus includes a storage unit that stores work instruction information including instructions of work procedures for instructing workers to perform predetermined work processes and work capacity model information including a plurality of models determined based on physical characteristics of the workers in respective work processes; a work sensing processing unit that senses the physical characteristic of the worker during a work that the worker performs based on the work instruction information and generates sensing information; and a work capacity evaluation processing unit that selects, for each of the workers, one or a plurality of models corresponding to the sensing information from the work capacity model information and associates the selected model with the sensing information to generate work capacity evaluation information.

### TECHNICAL FIELD

The present invention relates to a work support apparatus, a work support system, and a work support method.

### BACKGROUND ART

Patent Literature 1 discloses a production management apparatus that manages a production line involving a process in which a work is performed by a worker. The production management apparatus includes an activity state obtaining unit that obtains information indicating an activity state of the worker during a work, a first estimation unit that estimates emotion and cognition of the worker during a work based a primary indicator that uses the obtained information indicating the activity state and first learning data indicating a relationship between the activity state of the worker and the emotion of the worker and a relationship between the activity state of the worker and the cognition of the worker, a second estimation unit that estimates productivity of the worker based on a secondary indicator that uses the estimated emotion and cognition and second learning data indicating a relationship between the productivity of the worker and the emotion of the worker and a relationship between the productivity of the worker and the cognition of the worker, and an intervention determination unit that determines intervention timing and content to be provided for the worker based on the productivity estimated by the second estimation unit and a predetermined intervention condition.

### CITATION LIST

### Patent Literature

PTL 1: JP-A-2018-142258

### SUMMARY OF INVENTION

### Technical Problem

A technique disclosed in Patent Literature 1 describes an intervention method for a worker at a time point when emotion and cognition of the worker are respectively estimated based on biological measurement data and motion measurement data, productivity of the worker is further estimated, and a change amount of the productivity exceeds a threshold. An object of the technique is to prevent the productivity of the worker from lowering, and the technique cannot perform a work capacity analysis for improving the productivity of the worker.

An object of the invention is to perform a work capacity analysis for improving productivity of a worker.

### Solution to Problem

The present application includes a plurality of units for solving at least a part of the problems described above. An example of the units is as follows.

According to an aspect of the invention, a work support apparatus includes a storage unit that stores work instruction information including instructions of work procedures for instructing workers to perform predetermined work processes and work capacity model information including a plurality of models determined based on physical characteristics of the workers in respective work processes; a work sensing processing unit that senses the physical characteristic of the worker during a work that the worker performs based on the work instruction information and generates sensing information; and a work capacity evaluation processing unit that selects, for each of the workers, one or a plurality of models corresponding to the sensing information from the work capacity model information and associates the selected model with the sensing information to generate work capacity evaluation information.

### Advantageous Effect

According to the invention, a technique that can perform a work capacity analysis for improving productivity of a worker can be provided.

Problems, configurations, and effects other than those described above will become apparent from the following description of embodiments.

### BRIEF DESCRIPTION OF DRAWINGS

FIG. 1 is a diagram showing a configuration example of a work support apparatus according to a first embodiment.

FIG. 2 is a diagram showing an example of a data structure of process information.

FIG. 3 is a diagram showing an example of a data structure of work instruction information.

FIG. 4 is a diagram showing an example of a data structure of production line information.

FIG. 5 is a diagram showing an example of a data structure of sensing information.

FIG. 6 is a diagram showing an example of a data structure of work capacity model information.

FIG. 7 is a diagram showing an example of a data structure of work capacity evaluation information.

FIG. 8 is a diagram showing an example of a data flow.

FIG. 9 is a diagram showing an example of a usage form of the work support apparatus in a production line.

FIG. 10 is a diagram showing an example of a usage form of the work support apparatus in a plurality of target production lines.

FIG. 11 is a diagram showing an example of a hardware configuration of the work support apparatus.

FIG. 12 is a diagram showing an example of a flow of a work sensing processing.

FIG. 13 is a diagram showing an example of a flow of a work capacity evaluation processing.

FIG. 14 is a diagram showing an example of a flow of a process allocation adjustment processing.

FIG. 15 is a diagram showing an example of a flow of a work instruction generation processing.

FIG. 16 is a diagram showing an example of a combination of a granularity and a type of work instructions.

FIG. 17 is a diagram showing an example of work instructions having different granularities.

FIG. 18 is a diagram showing an example of a combination of a granularity and a type of work instructions.

FIG. 19 is a diagram showing a change example of a process allocation and a work instruction.

### DESCRIPTION OF EMBODIMENTS

In the following embodiments, a description may be divided into a plurality of sections or embodiments if necessary for convenience. Unless particularly specified, the sections or embodiments are not independent of each other, but have a relationship in which one section or embodiment is a variation, a detailed description, a supplementary description, or the like of a part or all of another section or embodiment.

In the following embodiments, when the number and the like (including the number of articles, a numeric value, a quantity, a range, and the like) of an element is mentioned, the parameters are not limited to specific numbers, and may be equal to or larger than the specific numbers, unless particularly specified or unless the parameters are apparently limited to the specific numbers in principle.

In the following embodiments, it is needless to say that constituent elements (including element steps and the like) are not necessarily essential, unless particularly specified and considered to be apparently essential in principle.

Similarly, in the following embodiments, shapes, position relationships, and the like of constituent elements and the like include those substantially approximate or similar to the shapes or the like unless otherwise particularly specified and considered to be unclear in principle. The same applies to the numerical values and the range described above.

In all drawings showing the embodiments, the same members are denoted by the same reference numerals in principle, and repetitive descriptions thereof will be omitted. However, the same member may be denoted by different reference numerals or names in a case in which confusion may occur when the same member shares a reference numeral or name with a member before a change such as an environmental change. Hereinafter, embodiments of the invention will be described with reference to the drawings.

A work support system that supports a manual work for a worker in a factory is provided. An example of a function of the work support system includes displaying a work instruction for each process on a display device provided at a work booth in an assembly work including a plurality of continuous processes. A worker can proceed to work in the work booth and operate an input device such as a touch panel at the time of completing each of the processes (corresponding to a process separation work), so that the worker can request the work support system to display a work instruction for a subsequent process.

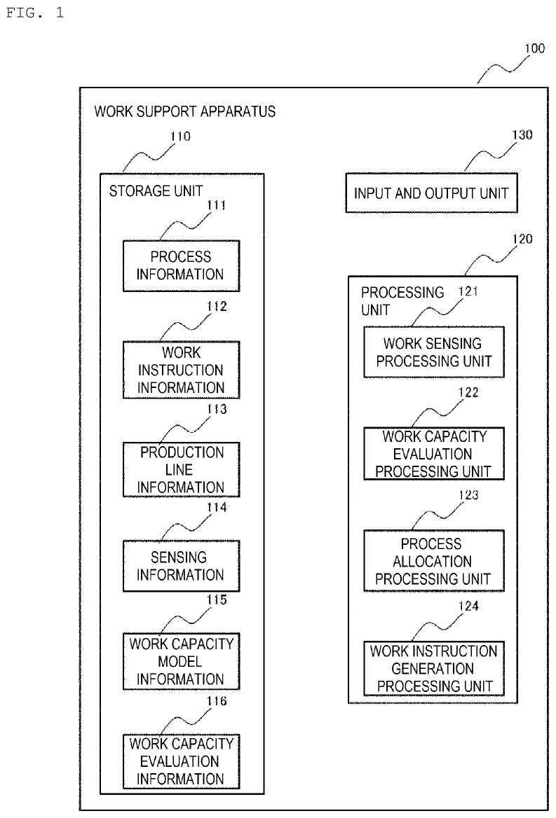

FIG. 1 is a diagram showing a configuration example of a work support apparatus according to a first embodiment. A work support apparatus 100 includes a storage unit 110, a processing unit 120, and an input and output unit 130. The storage unit 110 stores process information 111, work instruction information 112, production line information 113, sensing information 114, work capacity model information 115, and work capacity evaluation information 116. The processing unit 120 includes a work sensing processing unit 121, a work capacity evaluation processing unit 122, a process allocation processing unit 123, and a work instruction generation processing unit 124. The work support apparatus 100 may include a communication unit (not shown) that communicates with another device via a network such as a local area network (LAN).

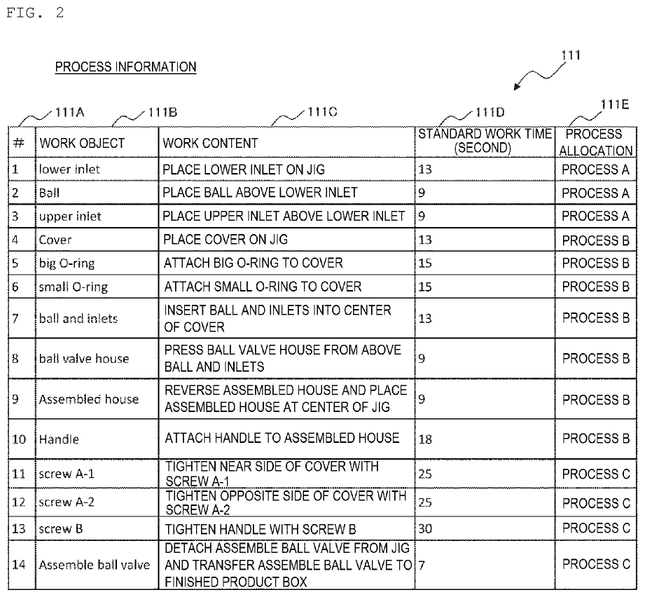

FIG. 2 is a diagram showing an example of a data structure of the process information. The process information 111 is a set of a series of works indicating a progress of production activities until a predetermined product is completed. For example, the process information 111 includes process information for assembling a ball valve. The process information 111 includes a work order 111A in which predetermined work procedures are ordered, a work object 111B indicating a target part in a work procedure, a work content 111C indicating a work action in language expression, standard work time 111D required by a standard worker, and a process allocation 111E.

In addition, the process information 111 may include jig data or tool data required in a work as an independent item. The process information 111 may include an attention item (such as a know-how item and a sensory tip) relating to efficiency or quality of a work as an item independent of the work content 111C.

The process information 111 may be associated with a separate association table that defines a new work procedure obtained by integrating (increasing a granularity) a plurality of work procedures into a single work procedure to integrate the work object or work content. In contrast, the process information 111 may be associated with a separate association table that defines a new work procedure obtained by dividing (reducing a granularity) a work content into more details.

The process allocation 111E is information for specifying a work group in order to function effectively when a work sharing policy is adopted so as to allocate all work procedures to a plurality of processes and perform works by a plurality of workers or in a plurality of work cells.

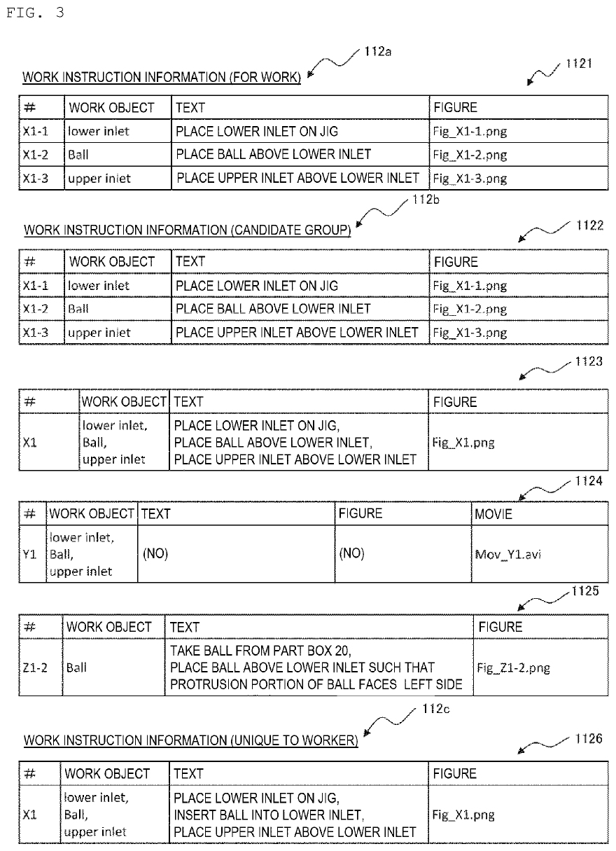

FIG. 3 is a diagram showing an example of a data structure of the work instruction information. The work instruction information 112 includes three types of data such as work instruction information (for a work) 112a, work instruction information (a candidate group) 112b, and work instruction information (unique to a worker) 112c.

The work instruction information (for a work) 112a includes a default table 1121 that is provided for a process allocated to a target worker when a work in the process is performed. The table 1121 includes information of a work number, a work object, a text instructing an action, and a figure of an action. The text and the figure serve as specific contents of a work instruction. These pieces of information are displayed on a work instruction screen in order of work numbers, and a worker performs a work while visually checking the information.

The work instruction information (a candidate group) 112b is an example of a candidate group of work instruction information that can be replaced with the work instruction information (for a work) 112a. A table 1122 is an example of information including the same data items and data as the table 1121. A table 1123 is an example of data obtained by integrating three work procedures recorded in the table 1121 into one work procedure. Although a work granularity of a work procedure “X1” in the table 1123 is large by integration, a text content is an example in which the three work procedures are simply added and no information is omitted or added.

A table 1124 is an example in which three work procedures are integrated into one work procedure in a similar manner to the table 1123, but is different from the table 1123 in that the table 1124 includes movie information for instructing a work content instead of the text and the figure. A table 1125 is an example in which a work number of “Z1-2” is an alternative to a work number of “X1-2” in the table 1122. In the work number of “Z1-2”, a more detailed instruction content (for example, specify a direction of a member and specify a storage position of a member) is provided for one work procedure. The detailed instruction often has an effect of contributing to quality assurance. In contrast, time required to read the text is increased, which may negatively affect work efficiency. When a detailed instruction is repeatedly given, motivation of a worker may be lowered.

Data such as a text, a figure, a two-dimensional movie, a three-dimensional movie, augmented reality (AR), virtual reality (VR), a voice, a smell, electrical stimulation, and pressure sensitivity, may be stored as types of work instructions in the work instruction information 112.

The work instruction information (unique to a worker) 112c is an example of work instruction information that is selected uniquely to a worker and that is generated by the work instruction generation processing unit 124 from a table selected from the work instruction information (a candidate group) 112b or generated by combining, when a plurality of tables are selected, the plurality of tables. In the present example, a table 1126 is the same as the table 1123 in the work instruction information (a candidate group) 112b. The work instruction information (for a work) 112a, the work instruction information (a candidate group) 112b, and the work instruction information (unique to a worker) 112c may include an attention item (such as a know-how item and a sensory tip) relating to efficiency or quality of a work as an independent item.

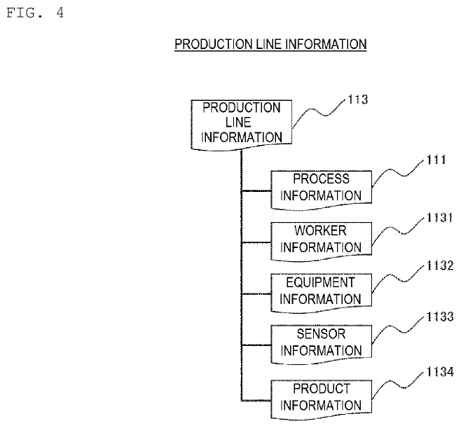

FIG. 4 is a diagram showing an example of a data structure of the production line information. The production line information 113 includes the process information 111, worker information 1131, equipment information 1132, sensor information 1133, and product information 1134. The worker information 1131 includes data indicating an attribute of a worker. For example, the worker information 1131 includes a unique number for specifying a worker, a name of a worker, age, gender, physical characteristics such as height, weight, a length of an arm, a size of a hand, a dominant hand, and a resting heart rate, experience values of a work and a related work, and psychological characteristics such as average motivation for a work.

The equipment information 1132 is information on equipment used in a work procedure. For example, the equipment information 1132 includes information for specifying a tool or a machine used in a production line and information indicating a state of the tool or the machine, such as an arrangement position of the tool or the machine, performance, a consumables list and remaining amount or replacement time.

The sensor information 1133 includes information indicating a type of a sensor used in a production line, particularly a type of a sensor used to detect physical characteristics such as a motion sensor or a camera for a worker, and an attachment position and a detection result of the sensor. The sensor information 1133 may be regarded as a part of equipment and included in the equipment information 1132.

The product information 1134 is information for specifying a target product produced on a production line. The process information 111 may be overwritten and updated by the process information 111 in an allocation result generated by the process allocation processing unit 123 at the time of producing a subsequent product.

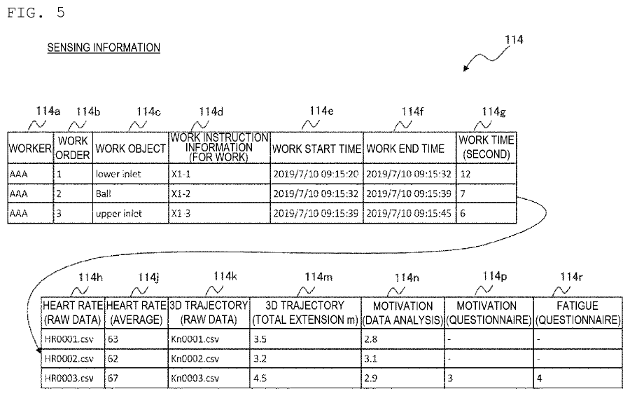

FIG. 5 is a diagram showing an example of a data structure of the sensing information. For a worker 114a, the sensing information 114 stores work instruction information 114d, work start time 114e, work end time 114f, work time 114g, various types of data of physical characteristics of a worker that are detected by a sensor during the work time, and various types of data of psychological characteristics of a worker during the work time that are associated with each of a work order 114b and a work object 114c.

In the present example, as the data of physical characteristics, a heart rate (raw data) 114h of a worker that is acquired by a heart rate monitor, a heart rate (average) 114j, a 3D trajectory (raw data) 114k that is an action trajectory acquired by a 3D camera, and a 3D trajectory (total extension m) 114m are associated with each of the work order 114b and the work object 114c in a comma separated value (CSV) file format.

In the present example, as the data of psychological characteristics, motivation (a data analysis) 114n, motivation (a questionnaire) 114p, and fatigue (a questionnaire) 114r are associated with each of the work order 114b and the work object 114c in a CSV file format.

The motivation (a data analysis) 114n is information obtained by analyzing the psychological characteristics of a worker and data acquired from the heart rate. For example, the motivation (a data analysis) 114n is information of converting, into predetermined index values, pleasant and unpleasant psychological states that are obtained as estimation results by using a Russell annular model and using the heart rate as an input.

The motivation (a questionnaire) 114p is a result obtained by recording psychological characteristics on a predetermined piece of paper by a worker after the worker performs each work procedure or inputting the psychological characteristics by performing a predetermined action. The fatigue (a questionnaire) 114r is also information obtained by receiving, in a similar manner, an input of fatigue felt by the worker.

The sensing information 114 is not limited to data described above, and may store information of other physical characteristics and information of other psychological characteristics in association with each other. Further, when the sensing information 114 includes a plurality of items indicating the same characteristic such as the motivation (a data analysis) 114n and the motivation (a questionnaire) 114p, an average value of values of the items may be calculated and recorded. Alternatively, a predetermined index value may be calculated and recorded by preferentially weighting information obtained directly from a worker, such as a questionnaire.

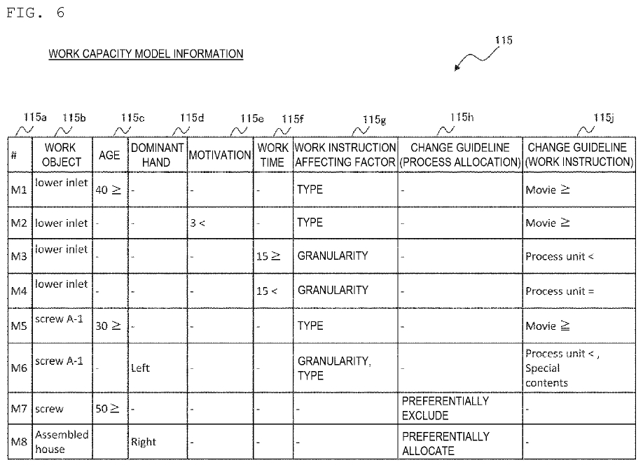

FIG. 6 is a diagram showing an example of a data structure of the work capacity model information. The work capacity model information 115 stores age 115c, a dominant hand 115d, motivation 115e, work time 115f, a work instruction affecting factor 115g that affects improvement of a capacity, a change guideline (a process allocation) 115h, and a change guideline (a work instruction) 115j that are associated with each work object 115b associated with a model number 115a. That is, the model number 115a is assigned to each work capacity model for each work object. The association is not limited to the age 115c, the dominant hand 115d, and the work time 115f, and other physical characteristics such as a heart rate and brain waves may be associated. The association is not limited to the motivation 115e, and other psychological characteristics such as fatigue may be associated.

That is, it can be said that the work capacity model information 115 stores one or a plurality of work capacity models in association with the work object 115b. Therefore, it can be said that the work capacity model information 115 includes a plurality of models that are determined based on physical characteristics of a worker in each work process.

Further, one or more work instruction change guidelines are associated with each work capacity model. A condition that satisfies a work instruction type or a work instruction granularity is defined by a predetermined description rule in the change guideline (a work instruction) 115j. The condition includes, for example, “movie ≥” (shift to an abundant instruction side where a type is a movie or an information amount is equal to or larger than a movie), “process unit <” (shift to a detailed instruction side where a granularity is smaller than a process unit), “process unit =” (a granularity is a process unit only), and “process unit <, special contents” (shift to a detailed instruction side where a granularity is smaller than a process unit and adopt a special work instruction (such as a left dominant hand)). It can be said that the change guideline (a work instruction) 115j defines a direction for changing a factor according to the work instruction affecting factor 115g.

Take a model “M1” in FIG. 6 as an example, for a work of “lower inlet” in the work object 115b, capacity improvement of a worker whose age 115c is “40 years old or elder” is affected by a “type” in the work instruction affecting factor 115g, and the change guideline (a work instruction) 115j presents to shift to a side indicating an instruction including a movie or an information amount equal to or larger than the movie. A model “M3” in FIG. 6 is modeled to reflect that a worker whose work time 115f is “15 seconds or more” is a worker who needs a specific and detailed instruction to smoothly perform a work. In the model “M3”, the change guideline (a work instruction) 115j presents to shift to a detailed instruction side where a granularity is smaller than a process unit. Similarly, a model “M4” in FIG. 6 is modeled to reflect that a worker whose work time 115f is “less than 15 seconds” is a worker who does not need a specific and detailed instruction to smoothly perform a work. In the model “M4”, the change guideline (a work instruction) 115j presents that a granularity is set to a largest and roughest process unit.

For example, a model “M7” in FIG. 6 is modeled to reflect that a worker whose age 115c is “50 years old or elder” is not good at screwing. In the model “M7”, the change guideline (a process allocation) 115h presents that the worker is preferentially excluded from the work if possible. In a model “M8” in FIG. 6, for a product structure of a part whose work object 115b is “assembled house”, work quality using a right dominant hand is higher than work quality using a left dominant hand. The change guideline (a process allocation) 115h presents to preferentially allocate the work to a worker whose dominant hand 115d is “right”.

A work capacity model may be configured according to a combination of a plurality of characteristics. The work capacity model is not limited to data in a table format, and may be defined as a formulated formula or parameters of the formula. A work object is not strictly the same work, and similar works may be classified, registered, and referred to. The work capacity model may be constructed based on sensing and work results in prior basic experiments or a previous work for a similar product. In a case where a work is repeatedly performed, when a deviation between the work capacity model and a result of the sensing information 114 acquired by the work sensing processing unit 121 is equal to or larger than a predetermined value, the work capacity evaluation processing unit 122 may update the model at any time by using the result of the sensing information 114.

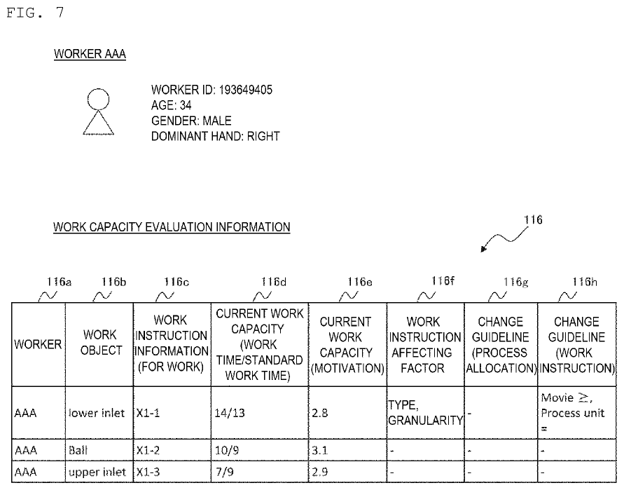

FIG. 7 is a diagram showing an example of a data structure of the work capacity evaluation information. The work capacity evaluation information 116 stores a current work capacity (work time/standard work time) 116d, a current work capacity (motivation) 116e, a work instruction affecting factor 116f, a change guideline (a process allocation) 116g, and a change guideline (a work instruction) 116h that are associated with each of a worker 116a, a work object 116b, and work instruction information (for a work) 116c.

FIG. 7 shows an example based on a result of sensing with a worker “AAA” as a target whose worker ID is 194649405, whose age is 34, whose gender is male and whose dominant hand is the right hand. For “lower inlet” in the work object 116b, work time/standard work time and motivation are calculated as a current work capacity. The current work capacity is not limited thereto, calculation of the motivation may be omitted and only the work time may be calculated as the current work capacity. Alternatively, psychological characteristics other than motivation, such as fatigue, and physical characteristics such as a height and a dominant hand may be extracted as a work capacity.

Here, when focusing on the current work capacity (motivation) 116e, an example of the psychological characteristics includes a value of the motivation (a data analysis) 114n in the sensing information 114.

Information obtained by integrating information in corresponding models is stored as the work instruction affecting factor 116f, the change guideline (a process allocation) 116g, and the change guideline (a work instruction) 116h. For example, since the age of the worker “AAA” is “40 years old or younger”, which does not correspond to “M1” and “M7” among models in FIG. 6, “M1” and “M7” are not extracted as the work capacity evaluation information 116. Similarly, the model “M3” is not extracted since the work time is “14”, and models “M5”, “M6”, and “M8” are not extracted since a work object “lower inlet” does not match those in the models “M5”, “M6”, and “M8”.

As a result, the work instruction affecting factor 116f, the change guideline (a process allocation) 116g, and the change guideline (a work instruction) 116h that correspond to the target object “lower inlet” of the worker “AAA” are respectively “type, granularity”, “no”, and “movie ≥, process unit =” in which the corresponding models “M2” and “M4” are integrated.

Referring back to FIG. 1, the work sensing processing unit 121 senses physical characteristics in a work for a worker who performs the work based on the work instruction information 112 and generates the sensing information 114. The work sensing processing unit 121 receives an input of psychological characteristics of the worker in each work process and stores the psychological characteristics in the sensing information. The work sensing processing unit 121 calculates an index value indicating a predetermined psychological characteristic by using physical characteristics of the worker in each work process, and stores the index value in the sensing information.

The work capacity evaluation processing unit 122 selects, for each worker, one or a plurality of models corresponding to the sensing information 114 from the work capacity model information 115, and generates the work capacity evaluation information 116 in association with the sensing information 114. When a deviation between physical characteristics in the sensing information 114 and physical characteristics in a model is equal to or larger than a predetermined value, the work capacity evaluation processing unit 122 changes the physical characteristics in the model, that is, updates a value of an item having a large deviation in the work capacity model information 115. Model information that extremely deviates from reality can be appropriately optimized.

The process allocation processing unit 123 changes an allocation of work processes performed by workers by using the sensing information 114 included in the work capacity evaluation information 116 and a work process order included in the process information 111 and outputs process allocation information. The process allocation processing unit 123 changes an allocation of work processes performed by workers by using a predetermined index value of the sensing information 114 included in the work capacity evaluation information 116 and a work process order included in the process information 111 and outputs the process allocation information.

The work instruction generation processing unit 124 specifies, for each worker, either a granularity or a type, or both a granularity and a type of an instruction of a work procedure in the work instruction information 112 according to a change guideline and generates the work instruction information 112. The work instruction generation processing unit 124 generates the work instruction information 112 when an approval for work instruction information specified according to the change guideline is obtained from a worker. The work instruction generation processing unit 124 generates the work instruction information 112 in which, for each worker, either a granularity or a type, or both a granularity and a type are changed in a predetermined cycle according to another change guideline that is different from the change guideline for the worker. The work instruction generation processing unit 124 may store the work instruction information 112 specified according to the change guideline in the storage unit 110, and perform respective processings of the work sensing processing unit 121 and the work capacity evaluation processing unit 122 again.

The input and output unit 130 controls an input into and an output from the work support apparatus 100.

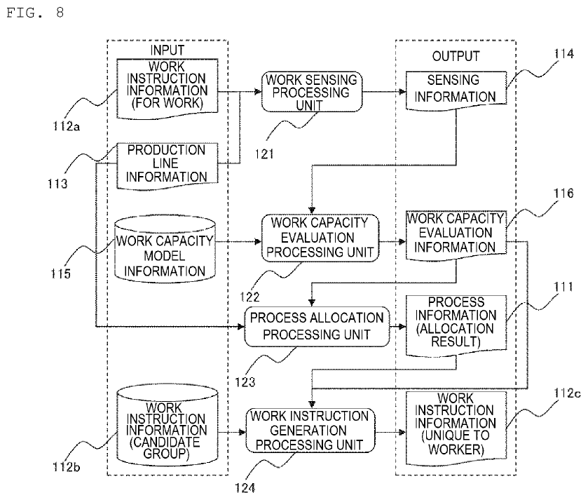

FIG. 8 is a diagram showing an example of a data flow. As shown in FIG. 8, the work sensing processing unit 121 performs a processing by using the work instruction information (for a work) 112a and the production line information 113 as inputs, and outputs the sensing information 114 as intermediate data.

The work capacity evaluation processing unit 122 performs a processing by using the sensing information 114 and the work capacity model information 115 as inputs, and outputs the work capacity evaluation information 116 as intermediate data.

The process allocation processing unit 123 performs a processing by using the production line information 113 and the work capacity evaluation information 116 as inputs, and outputs the process information 111 as an allocation result.

The work instruction generation processing unit 124 performs a processing by using, as inputs, the work capacity evaluation information 116, the work instruction information (a candidate group) 112b, and the process information 111 serving as an allocation result, and outputs the work instruction information (unique to a worker) 112c.

The data flow shown in FIG. 8 is an example, and may be different in a modification of the present embodiment. For example, the work sensing processing unit 121 outputs the sensing information 114 as intermediate data, but the sensing information 114 may be actualized as output information by displaying the sensing information 114 on a screen.

Alternatively, since the work instruction information (unique to a worker) 112c and the work instruction information (for a work) 112a that are shown as outputs have similar data structures, a processing of each processing unit may be cyclically performed for a plurality of times by using the work instruction information (unique to a worker) 112c as an input. Specifically, the work instruction generation processing unit 124 may store the work instruction information (unique to a worker) 112c specified according to a change guideline in the storage unit 110, and perform processings of the work sensing processing unit 121, the work capacity evaluation processing unit 122, and the process allocation processing unit 123 again.

The processings are continuously repeated in such a cyclical manner, so that sensing information can be accumulated, and output accuracy of each subsequent processing unit can be improved. The work instruction information 112 is updated automatically by repeatedly performing the processings, so that a work instruction suitable for a worker can be provided without manpower, and work efficiency can be improved without management man-hours.

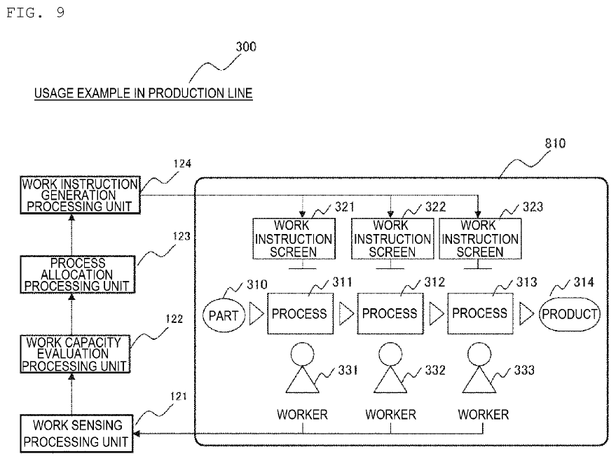

FIG. 9 is a diagram showing an example of a usage form of the work support apparatus in a production line. In a usage example 300 in a production line 810, an input part 310 is assembled in order of three processes 311, 312, and 313, and a product 314 is assembled. Workers 331, 332, and 333 are arranged in respective processes, and perform works while checking work instruction screens 321, 322, and 323 for teaching work contents.

Work situations of workers are sensed by the work sensing processing unit 121 and processed by the work capacity evaluation processing unit 122, the process allocation processing unit 123, and the work instruction generation processing unit 124 to change a process allocation at the time of assembling a subsequent product. Changed process information and work instruction information unique to a worker corresponding to a work capacity of each worker in a previous work are presented on the work instruction screens 321, 322, and 323. An effect of improving motivation of a worker, shortening a work learning period, and improving labor productivity can be obtained by a process allocation and a work instruction that are suitable for each worker.

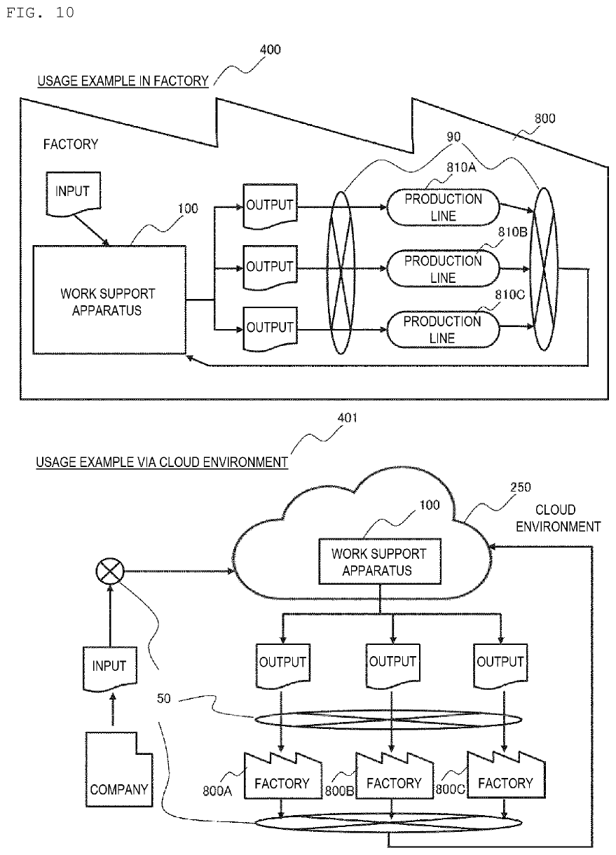

FIG. 10 is a diagram showing an example of a usage form of the work support apparatus in a plurality of target production lines. In a usage example 400 in a factory 800, the work support apparatus 100 that performs management inside the factory receives input information relating to production in the factory 800 via a communication unit, and outputs (transmits) process information and work instruction information suitable for production lines 810A, 810B, and 810C provided in the factory 800 to the production lines 810A, 810B, and 810C via a network 90 such as a local area network (LAN), so that production costs inside the factory can be reduced.

Operation performance of production lines and operation performance of equipment that constitutes a production line are collected via the network 90 and equipment information is updated based on the operation performance, so that an input of a process plan and an input of a work instruction device are values corresponding to the performance and a more realistic process plan and a more realistic work instruction can be made.

In a usage example 401 via a cloud environment 250, the work support apparatus 100 in the cloud environment 250 receives input information relating to production in all producible factories 800A, 800B, and 800C via a network 50 such as the Internet, and outputs (transmits) process information and work instruction information suitable for production lines provided in all of the factories 800A, 800B, and 800C to each factory or production line via the network 50, so that production costs can be optimized in consideration of production lines in all producible factories 800A, 800B, and 800C.

In each factory, operation performance of production lines and operation performance of equipment that constitutes a production line are collected via the network 50 and equipment information is updated based on the operation performance, so that an input of the work support apparatus is a value corresponding to the performance and a more realistic process plan and a more realistic work instruction can be made. All producible factories may include factories of an own company, factories of another company, and both factories of an own company and factories of another company. The work capacity model information 115 constructed and updated based on operation performance in a factory of the own company can be used in an operation in another factory of the own company or a factory of another company via the cloud environment 250.

In particular, when the work capacity model information 115 is applied to a factory of another company, only an optimized result of a process plan and a work instruction can be provided as a service without disclosing information of the own company. When a contract permits, the work capacity model information 115 can be updated based on operation performance of another company, and the sufficiently updated work capacity model information 115 can be used even when operation performance of the own company is small.

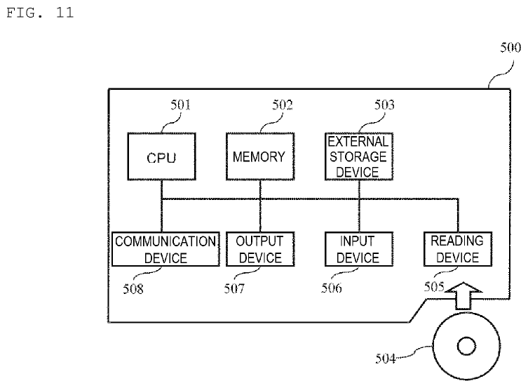

FIG. 11 is a diagram showing an example of a hardware configuration of the work support apparatus. The work support apparatus 100 can be implemented by a general computer 500 including a central processing unit (CPU) 501, a memory 502, an external storage device 503 such as a hard disk drive (HDD) and a solid state drive (SSD), a reading device 505 that reads information from and writes information into a portable storage medium 504 such as a compact disk (CD) and a digital versatile disk (DVD), an input device 506 such as a keyboard, a mouse, an acceleration sensor, and a heart rate sensor, an output device 507 such as a display, and a communication device 508 that communicates with another computer via a communication network such as the Internet. Alternatively, the work support apparatus 100 can be implemented by a network system including a plurality of computers 500.

For example, the processing unit 120 can be implemented by loading a predetermined program stored in the external storage device 503 into the memory 502 and executing the program by the CPU 501. The input and output unit 130 can be implemented by the CPU 501 using the input device 506 and the output device 507. The storage unit 110 can be implemented by the CPU 501 using the memory 502 or the external storage device 503.

The predetermined program for implementing the processing unit 120 may be downloaded into the external storage device 503 from the storage medium 504 via the reading device 505 or from a network via the communication device 508, and then loaded into the memory 502 and executed by the CPU 501. Alternatively, the program may be directly loaded into the memory 502 from the storage medium 504 via the reading device 505 or from the network via the communication device 508 and executed by the CPU 501. The work support apparatus 100 is not limited thereto, and may be a wearable computer worn on a worker, such as a headset, goggles, glasses, and an intercom.

### Work Sensing Processing

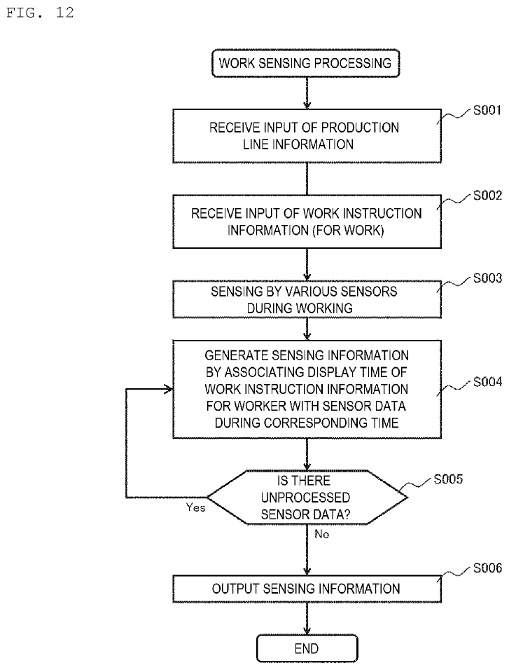

FIG. 12 is a diagram showing an example of a flow of a work sensing processing. The work sensing processing is started when the input and output unit 130 of the work support apparatus 100 receives a predetermined instruction.

First, the input and output unit 130 receives an input of the target production line information 113 (step S001). Since the number of engaged workers is different depending on a target production line, information of all target workers whose process allocation is to be changed is acquired. Alternatively, a worker who has been input in advance may be selectively associated with a production line. For example, in the example of the production line 810, there are three target workers 331, 332, and 333. Physical characteristics or psychological characteristics of the three workers and information of sensors to be used are associated with each other.

Then, the input and output unit 130 receives an input of the work instruction information (for a work) presented to a worker who has been input in step S001 (step S002).

Next, the work sensing processing unit 121 senses work situations by various sensors during working in a work performed by each worker based on work instruction information (step S003). Here, the various sensors include a heart rate monitor that measures a heart rate of a worker. However, the various sensors are not limited to the heart rate monitor, and may include a thermometer, a hygrometer, a seismograph, a microphone, and the like for measuring a work environment. The various sensors may include a two-dimensional or three-dimensional camera (for mainly acquiring information of physical characteristics) for measuring an action of a worker, a line-of-sight tracking for measuring a line of sight of a worker, an electroencephalograph (for mainly acquiring information of psychological characteristics), and the like.

The various sensors may include a laser sensor or a touch sensor that detects that apart is taken out of a part box, a physical button or a button on a screen for switching a work instruction screen, and the like. The various sensors may include a physical button or a button on a screen for obtaining an answer in a questionnaire for obtaining an intentional input of a psychological state of a worker.

Then, the work sensing processing unit 121 generates sensing information by associating display time of the work instruction information for each worker with sensor data acquired from the various sensors in a process during the corresponding time (step S004). Here, process information included in the work instruction information is also associated with the sensor data, so that sensing information of each process can be easily extracted in other processing units. The work sensing processing unit 121 may calculate an index value indicating a predetermined psychological characteristic by using physical characteristics of a worker in each work process, and the index value may be stored in the sensing information 114. For example, the work sensing processing unit 121 may specify motivation indicating a psychological characteristic from a heart rate which is a physical characteristic, and the motivation may be stored in the sensing information 114.

Next, the work sensing processing unit 121 determines whether there is unprocessed sensor data (step S005). If there is no unprocessed sensor data (“No” in step S005), the work sensing processing unit 121 advances a control to step S006. If there is unprocessed sensor data (“Yes” in step S005), the work sensing processing unit 121 returns the control to step S004. Since sensors used in a target production line are different, data of all used sensors may be acquired and sensing information may be integrated by associating the data of all used sensors with a work, work time, and data of other sensors.

Then, the input and output unit 130 outputs the sensing information 114 (step S006).

An example of the flow of the work sensing processing is described above. According to the work sensing processing, a detection value related to physical characteristics and psychological characteristics of a worker can be acquired for each work in a production line, and the detection value can be output as the sensing information.

### Work Capacity Evaluation Processing

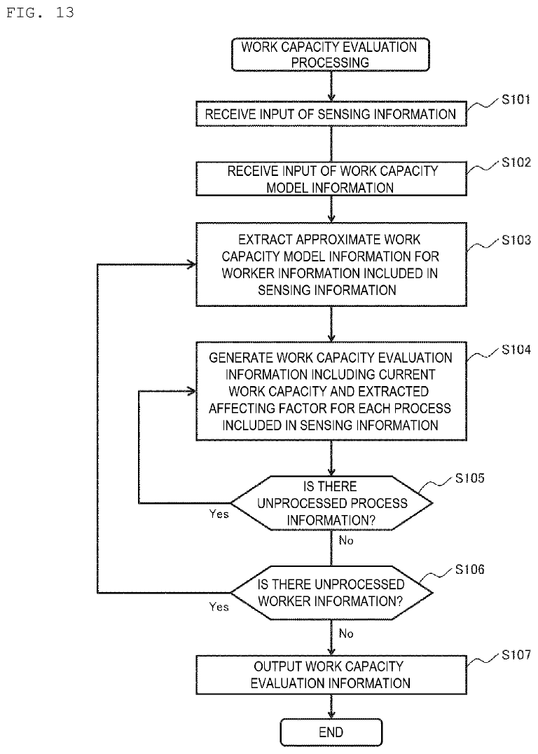

FIG. 13 is a diagram showing an example of a flow of a work capacity evaluation processing. The work capacity evaluation processing is started when the input and output unit 130 of the work support apparatus 100 receives a predetermined instruction.

First, the input and output unit 130 receives an input of the sensing information 114 generated by the work sensing processing unit 121 (step S101).

Then, the input and output unit 130 receives an input of the work capacity model information 115 stored in the storage unit 110 (step S102).

Next, the work capacity evaluation processing unit 122 extracts approximate work capacity model information from the work capacity model information 115 received in step S102 for one piece of worker information included in the sensing information received in step S101 (step S103).

Then, the work capacity evaluation processing unit 122 generates work capacity evaluation information including a current work capacity and an extracted affecting factor for each process included in the sensing information (step S104). Specifically, for each piece of process information included in the sensing information received in step S101, the work capacity evaluation processing unit 122 calculates a current work capacity for each worker by using the sensing information 114. The work capacity evaluation processing unit 122 specifies a factor affecting a work capacity in a process from the work capacity model information 115 extracted in step S103, and generates the work capacity evaluation information 116 including the current work capacity and the affecting factor by removing duplication and combining the current work capacity and the affecting factor (step S104).

In this step, the motivation (a data analysis) 114n and the motivation (a questionnaire) 114p in the sensing information 114 correspond to items having the same contents. When the work capacity evaluation information 116 is generated, any one of the items may be selected to generate the current work capacity (motivation) 116e, or the items may be integrated by calculating an average value of the items. A result of selecting the motivation (a data analysis) 114n is shown in the example of the worker AAA in FIG. 7.

Next, the work capacity evaluation processing unit 122 determines whether there is unprocessed process information (step S105). If there is no unprocessed process information (“No” in step S105), the work capacity evaluation processing unit 122 advances a control to step S106. If there is unprocessed process information (“Yes” in step S105), the work capacity evaluation processing unit 122 returns the control to step S104.

Then, the work capacity evaluation processing unit 122 determines whether there is unprocessed worker information (step S106). If there is no unprocessed worker information (“No” in step S106), the work capacity evaluation processing unit 122 advances the control to step S107. If there is unprocessed worker information (“Yes” in step S106), the work capacity evaluation processing unit 122 returns the control to step S103. The sensing information 114 may include a plurality of pieces of worker information. The work capacity evaluation information 116 can be generated for each worker by repeatedly performing the processing in such a manner.

Next, the input and output unit 130 outputs the work capacity evaluation information 116 (step S107). Specifically, the input and output unit 130 generates and displays screen information including the work capacity evaluation information 116 of each worker.

An example of the flow of the work capacity evaluation processing is described above. According to the work capacity evaluation processing, a work capacity analysis for improving productivity of a worker can be performed.

### Process Allocation Adjustment Processing

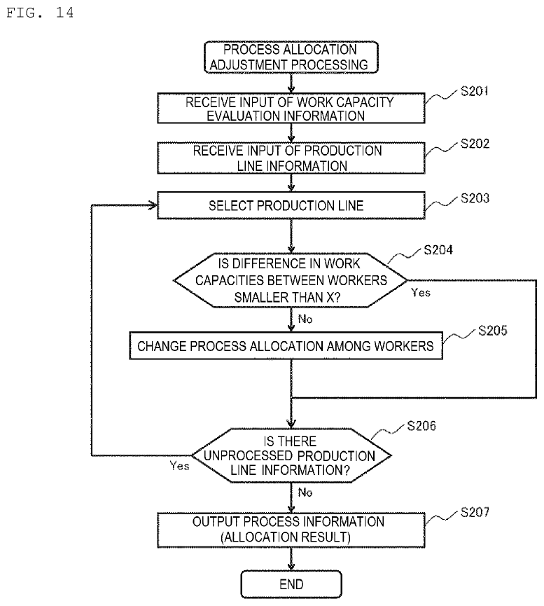

FIG. 14 is a diagram showing an example of a flow of a process allocation adjustment processing. The process allocation adjustment processing is started when the input and output unit 130 of the work support apparatus 100 receives a predetermined instruction.

First, the input and output unit 130 receives an input of the work capacity evaluation information generated by the work capacity evaluation processing unit 122 (step S201).

Then, the input and output unit 130 receives an input of the production line information 113 stored in the storage unit 110 (step S202). Here, the process information 111 in the production line information 113 is essential input information.

Next, the process allocation processing unit 123 selects one target production line of a process allocation (Step S203).

Then, the process allocation processing unit 123 determines whether a difference in work capacities between workers in the selected production line is smaller than a predetermined threshold (step S204). Specifically, the process allocation processing unit 123 respectively compares a current work capacity of each worker included in the work capacity evaluation information 116 with a work capacity of a worker in the production line, and determines whether a difference in work capacities between workers is smaller than a threshold X (step S204). If the difference in work capacities between workers is equal to or larger than the threshold X (“No” in step S204), the process allocation processing unit 123 advances a control to step S205. If the difference in work capacities between workers is smaller than the threshold X (“Yes” in step S204), the process allocation processing unit 123 advances the control to step S206.

Here, the threshold X may specify, for example, work time (seconds) as a work capacity. An evaluation value of a psychological characteristic such as motivation may be specified as a work capacity. An evaluation value obtained from a work result of a previous work such as work quality may be used as a work capacity. Further, an evaluation function such as a sum or a weighted sum of predetermined indexes that are different from each other may be defined, and an overall predetermined evaluation value may be specified as a work capacity. The threshold value X may be input by a user and received by the process allocation processing unit 123 via the input and output unit 130. The process allocation processing unit 123 may use, as the threshold X, a value that is stored in advance in the storage unit 110 as an initial value.

Next, the process allocation processing unit 123 changes a process allocation among workers (step S205). Here, the change in the process allocation refers to a change of adjusting processes allocated to workers so as to reduce a difference in work capacities among workers. For example, when the processes are adjusted by using a plurality of indexes such as work time and work quality, there may be a conflict in which improvement of any one of the indexes leads to deterioration of the other one index, or the like. In this case, the problem can be solved by using a method of defining a priority among items of the work capacity. Alternatively, the processes may be adjusted by using an evaluation function including a composite index.

In a process allocation changing processing, the process allocation processing unit 123 may specify a change guideline for a process allocation in which strengths and weaknesses are modeled according to characteristics of workers by referring to the work capacity evaluation information 116, and determine a prioritized allocation or exclude a worker from an allocation. In this case, the process allocation processing unit 123 may determine in advance whether to prioritize a change guideline or to prioritize an evaluation value of a work capacity according to a predetermined setting. Alternatively, the process allocation processing unit 123 may use a weighted value quantified based on an importance degree of each change guideline to perform quantitative evaluation according to an evaluation function in which the importance degree and the evaluation value of a work capacity are weighted and combined according to the weighted value.

Then, the process allocation processing unit 123 determines whether there is unprocessed production line information (step S206). If there is no unprocessed production line information (“No” in step S206), the process allocation processing unit 123 advances the control to step S207. If there is unprocessed production line information (“Yes” in step S206), the process allocation processing unit 123 returns the control to step S203.

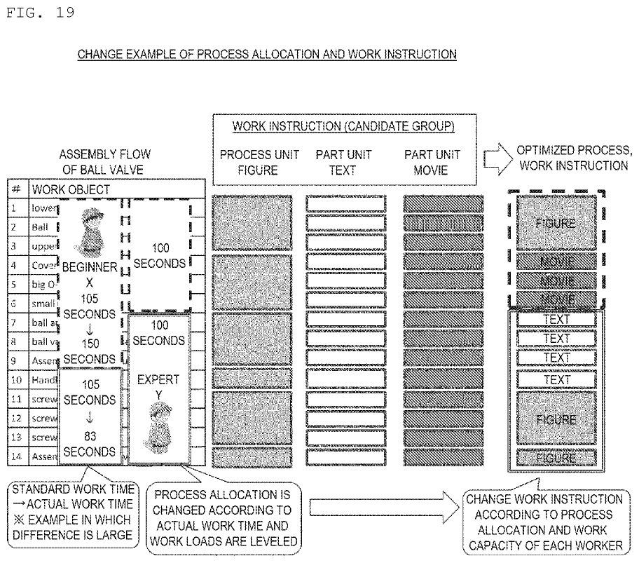

Next, the input and output unit 130 outputs the process information (an allocation result) 111 (step S207). Output information of the process information (an allocation result) 111 will be described later in detail using FIG. 19.

An example of the flow of the process allocation adjustment processing is described above. According to the process allocation adjustment processing, processes performed by workers can be adjusted based on a work capacity analysis result of the workers. For example, according to a change guideline of a process in a model that matches each worker, a worker who is not good at a work can be replaced by another worker, work processes of a beginner who is not familiar with a work can be reduced, and work processes of an expert can be increased, so that an adjustment of emphasizing completion of all works in a production line within standard work time can be performed.

### Work Instruction Generation Processing

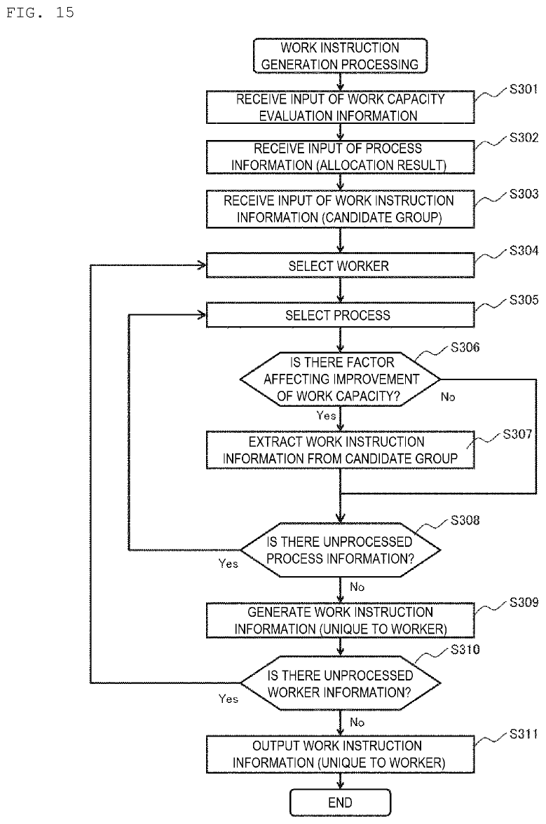

FIG. 15 is a diagram showing an example of a flow of a work instruction generation processing. The work instruction generation processing is started when the input and output unit 130 of the work support apparatus 100 receives a predetermined instruction.

First, the input and output unit 130 receives an input of the work capacity evaluation information 116 (step S301).

Then, the input and output unit 130 receives an input of the process information (an allocation result) 111 (step S302).

Next, the input and output unit 130 receives an input of the work instruction information (a candidate group) 112b stored in the storage unit 110 (step S303).

Then, the work instruction generation processing unit 124 selects one piece of worker information included in the work capacity evaluation information 116 that has been input in step S301 (step S304).

Next, the work instruction generation processing unit 124 selects one process of the process information (an allocation result) 111 included in the process information (an allocation result) that has been input in step S302 (step S305).

Then, the work instruction generation processing unit 124 determines whether a factor (work instruction affecting factor 116f) affecting improvement of a work capacity is included in the work capacity evaluation information 116 for the selected worker and the selected process (step S306). If an affecting factor is included (“Yes” in step S306), the work instruction generation processing unit 124 advances a control to step S307. If the affecting factor is not included (“No” in step S306), the work instruction generation processing unit 124 advances the control to step S308.

Next, the work instruction generation processing unit 124 extracts work instruction information including the factor affecting improvement of a work capacity from the work instruction information (a candidate group) 112b (step S307). Specifically, when a model including a “type” or a “granularity” corresponds to information of the work instruction affecting factor 116f of a worker, the work instruction generation processing unit 124 reads information of the change guideline (a work instruction) 116h, and changes the work instruction information (a candidate group) 112b according to the change guideline corresponding to the type and the granularity.

The change guideline stores a previous work instruction for a worker. An axis of a “type” in a direction of increasing an information amount from a “text”, a “figure (still image)”, a “movie” to “AR/VR” is assumed and a change is made along the axis. For example, if the change guideline is “movie ≥” (shift to an abundant instruction side where a type is a movie or an information amount is equal to or larger than a movie), a work instruction of a “text” and a “figure (still image)” is changed to one of a work instruction of a “movie” and a work instruction of “AR/VR” to which a change amount from the current work instruction is less and which has as less information amount as possible.

An axis of a “granularity” in a direction of reducing a granularity from “process”, “part” to “step” is assumed and a change is made along the axis. For example, if the change guideline is “Process unit <” (shift to a detailed instruction side where a granularity is smaller than a process unit), a work instruction of a “process” is changed to a work instruction of a “part” or “step”. In the example described above, a model corresponding to a worker is extracted and a granularity and a type of an instruction corresponding to a characteristic of the model are changed according to a change guideline, but the change in a granularity and a type is not limited thereto. Corresponding to a skill level of a worker, a granularity of an instruction may be gradually changed to be “large (rough)” and a type may be gradually changed in a direction of “reducing” an information amount.

Then, the work instruction generation processing unit 124 determines whether there is unprocessed process information in the process information (an allocation result) 111 (step S308). If there is no unprocessed process information (“No” in step S308), the work instruction generation processing unit 124 advances the control to step S309. If there is unprocessed process information in the process information (an allocation result) 111 (“Yes” in step S308), the work instruction generation processing unit 124 returns the control to step S305.

Next, the work instruction generation processing unit 124 generates the work instruction information (unique to a worker) 112c (step S309). This is because the work instruction information (unique to a worker) 112c for the worker selected in step S304 is changed in a target process based on the process information (an allocation result) 111. This is because when there is work instruction information extracted in step S307, the work instruction information (unique to a worker) 112c is also replaced by the extracted work instruction information.

Then, the work instruction generation processing unit 124 determines whether there is unprocessed worker information in the worker information included in the work capacity evaluation information 116 (step S310). If there is no unprocessed worker information (“No” in step S310), the work instruction generation processing unit 124 advances the control to step S311. If there is unprocessed worker information (“Yes” in step S310), the work instruction generation processing unit 124 returns the control to step S304.

Next, the input and output unit 130 outputs the work instruction information (unique to a worker) 112c (step S311).

An example of the flow of the work instruction generation processing is described above. According to the work instruction generation processing, corresponding to a skill level of a worker, a granularity of an instruction may be gradually changed to be “large (rough)” and a type may be gradually changed in a direction of “reducing” an information amount.

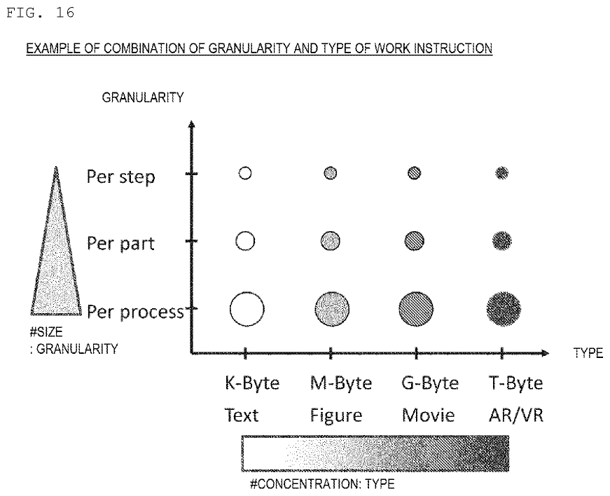

FIG. 16 is a diagram showing an example of a combination of a granularity and a type of a work instruction. FIG. 16 shows a mapping concept of a granularity and a type of a work instruction included in the work instruction information (a candidate group) 112b. A vertical axis indicates a granularity of the work instruction, and the granularity of works instructed at one time is reduced toward an upper direction in FIG. 16. For example, an instruction in which a plurality of parts to be processed in a process are collected and are continuously combined (a plurality of parts are combined) is presented in a process unit, while an instruction in which one instruction is limited to one part is presented in a part unit. Further, an instruction in which a work relating to a part is divided into more details is presented in a step unit (a work element unit). For example, a work is divided into three action units such as taking a part out of a part box, assembling the part, and confirming assembly.

A horizontal axis indicates a type of a work instruction, and includes a larger amount of information towards a right direction in FIG. 16. Typically, since an information amount included in a text, a figure, a movie, AR or VR, or the like increases, the information amount can also be indicated by a data size of the work instruction. Information in the work instruction information (a candidate group) 112b is arranged on an axis of a granularity and an axis of a type in this manner. Based on the work instruction affecting factor 116f shown in the work capacity evaluation information 116, the work instruction generation processing unit 124 extracts a plurality of combinations of work instructions from the work instruction information (a candidate group) 112b, and selects a combination having shortest predicted work time.

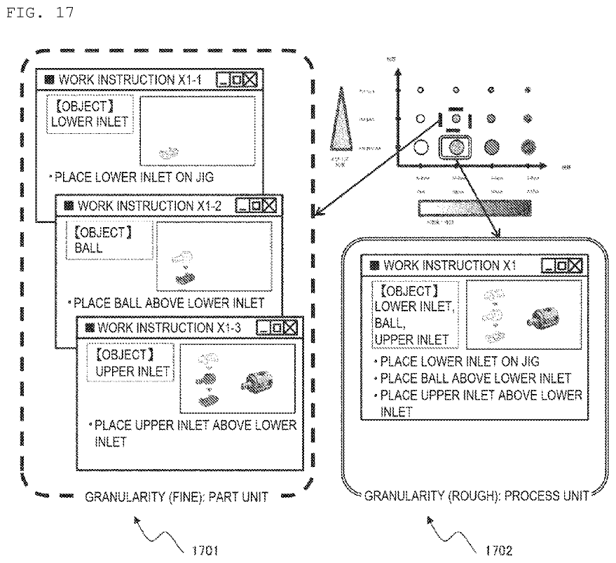

FIG. 17 is a diagram showing an example of work instructions having different granularities. A work instruction 1701 on a left side and a work instruction 1702 on a right side in FIG. 17 include both texts and figures as work contents, and have different granularities. A granularity is small (fine) in the work instruction 1701 on the left side and a granularity is large (rough) in the work instruction 1702 on the right side. In the example of the combination in FIG. 16, a granularity is a “part” and a type is a “figure” in the work instruction 1701 on the left side and a granularity is a “process” and a type is a “figure” in the work instruction 1702 on the right side.

In the work instruction 1701 on the left side in which a granularity of a work instruction is small, an assembly work is instructed in a part unit on three screens. On the other hand, in the work instruction 1702 on the right side in which a granularity of a work instruction is large, works for three parts are instructed by integrating the works for three parts into one work. Here, the work instruction 1701 on the left side corresponds to the table 1122 of the work instruction information (a candidate group) 112b and the table 1121 of the work instruction information (for a work) 112a, and the work instruction 1702 on the right side corresponds to the table 1123 of the work instruction information (a candidate group) 112b and the table 1126 of the work instruction information (unique to a worker) 112c.

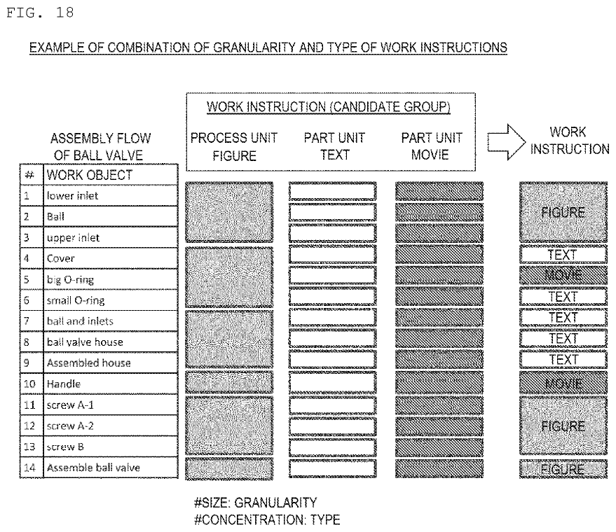

FIG. 18 is a diagram showing an example of a combination of a granularity and a type of work instructions. A candidate group of a combination of a granularity and a type of work instructions is held in an assembly flow of a ball valve. For example, for working objects having work orders of “1 to 3”, information corresponding to an individual work order is held in a text (a part unit) and a movie (a part unit). On the other hand, information on the work orders of “1 to 3” is collected as one figure and held in a figure (a process unit). A work capacity of a worker can be improved by combining information according to a work capacity and generating and presenting a new work instruction. For example, a figure (a process unit) is shown as a work procedure for the work objects having work orders of “1 to 3”, a text (a part unit) is shown as a work procedure for a work object having a work order of “4”, and a movie (a part unit) is shown as a work procedure for a work object having a work order of “5”, so that a work instruction suitable for a model of a worker can be generated.

FIG. 19 is a diagram showing a change example of a process allocation and a work instruction. The present example is an example focusing on work time as an evaluation axis of a work capacity. In the present example, a ball valve assembly including a total of 14 processes is allocated to two workers (a beginner X and an expert Y). In a standard work, the beginner X is in charge of work orders “1 to 9” and the expert Y is in charge of work orders “10 to 14”. A sum of standard work time of work orders that are in the charge of respective workers is 105 seconds (refer to standard work time 111D in FIG. 2). However, there is a large difference in work capacities between the beginner X and the expert Y. Actual work time of the beginner X is 150 seconds and actual work time of the expert Y is 83 seconds.

On such a basis, a process allocation is changed according to actual work time of each worker and work loads are leveled in a process allocation adjustment processing. As a result, the process allocation is changed to a process allocation in which the beginner X is in charge of work orders “1 to 6” and the expert Y is in charge of work orders “7 to 14”. In a work instruction generation processing, a type having an abundant information amount such as a movie and a figure is used for the beginner X and a granularity of the type is fine. A type having a small information amount such as a text and a figure is used for the expert Y and a granularity of the type is rough.

In the present example, a process allocation is optimized by focusing on work time only. Alternatively, various processings such as a processing of focusing on a physical characteristic of a worker, in which if there is a process that is difficult for a worker having a left dominant hand, the process is avoided to be allocated to the worker having a left dominant hand, and a processing of focusing on a physical characteristic of a worker, in which if there is process that improves motivation of a worker, the process is preferentially allocated to the worker as compared with other workers, may be performed in the process allocation adjustment processing.

An embodiment of the work support apparatus according to the invention has been described above. According to the work support apparatus according to the embodiment of the invention, a work capacity analysis for improving productivity of a worker can be performed. Accordingly, a work support apparatus that improves motivation of a worker, shortens a work learning period, and improves labor productivity can be provided.

Here, for example, in a case where it is desired to change only a process allocation without changing a work instruction, the work instruction generation processing unit 124 may be omitted and a work instruction generation processing may not be performed. Alternatively, in a case where it is desired to change a work instruction only without changing a process allocation, the process allocation processing unit 123 may be omitted and a process allocation adjustment processing may not be performed.

For example, the work instruction generation processing unit 124 may receive an approval for changing a work instruction from a worker before a work instruction generation processing. Accordingly, a change reflecting an intention of a worker can be made. Since the change is notified in advance and an approval is obtained from a worker, the worker can feel comfortable to perform a work.

For example, the work instruction generation processing unit 124 may change a type and a granularity of a work instruction in a regular or random cycle to issue a work instruction regardless of a current work capacity of a worker. In this case, a granularity of a work instruction and a type of a work instruction are required to change in a direction in which the granularity of a work instruction is reduced and a direction in which the type of a work instruction has an abundant information amount so that a work can be performed. In this manner, a certain level of tension stimulation can be given to a worker and attention or motivation of a worker can be maintained. Since such psychological characteristics may appear later, it may be effective to execute a processing in advance.

The embodiment described above has been described in detail for easy understanding of the invention, and the invention is not necessarily limited to include all configurations described above.

A part of configurations of the embodiment can be added, deleted, or replaced. Units, configurations, functions, processing units, and the like described above may be partially or entirely implemented by hardware such as through design using an integrated circuit. The units, configurations, functions, and the like described above may be implemented by software by a processor interpreting and executing programs for implementing respective functions. Information such as a program, a table, or a file for implementing each function can be stored in a recording device such as a memory, a hard disk, and an SSD, or a recording medium such as an IC card, an SD card, or a DVD.

Control lines and information lines according to the embodiment described above indicate what is considered necessary for the description, and not all of the control lines and the information lines are necessarily shown in a product. In practice, it may be considered that almost all of the configurations are connected to each other.

As described above, the invention has been described centering on the embodiment.

### REFERENCE SIGN LIST

100 work support apparatus
110 storage unit
111 process information
112 work instruction information
113 production line information
114 sensing information
115 work capacity model information
116 work capacity evaluation information
120 processing unit
121 work sensing processing unit
122 work capacity evaluation processing unit
123 process allocation processing unit
124 work instruction generation processing unit
130 input and output unit

1. A work support apparatus comprising:
a storage unit that stores work instruction information including instructions of work procedures for instructing workers to perform predetermined work processes and work capacity model information including a plurality of models determined based on physical characteristics of the workers in respective work processes;
a work sensing processing unit that senses the physical characteristic of the worker during a work that the worker performs based on the work instruction information and generates sensing information; and
a work capacity evaluation processing unit that selects, for each of the workers, one or a plurality of models corresponding to the sensing information from the work capacity model information and associates the selected model with the sensing information to generate work capacity evaluation information.

2. The work support apparatus according to claim 1, wherein
the storage unit stores process information in which the work processes are ordered, and
the work support apparatus further comprises a process allocation processing unit that changes an allocation of the work processes performed by the workers, by using the sensing information included in the work capacity evaluation information and the work process order included in the process information, and outputs process allocation information.

3. The work support apparatus according to claim 1, wherein
a change guideline of the instruction of the work procedure in the work instruction information is associated with the model of the work capacity evaluation information, and
the work support apparatus further comprises a work instruction generation processing unit that specifies, for each of the workers, either a granularity or a type, or both a granularity and a type of the instruction of the work procedure in the work instruction information according to the change guideline and generates the work instruction information.

4. The work support apparatus according to claim 1, wherein
the work process is a process relating to a part assembly work, and
a granularity of the instruction of the work procedure in the work instruction information includes at least a granularity shown in a part unit of a work object, a granularity obtained by dividing a granularity in a part unit into a plurality of granularities, and a rough granularity obtained by combining a plurality of granularities in a part unit.

5. The work support apparatus according to claim 1, wherein
the work process is a process relating to a part assembly work, and
a type of the instruction of the work procedure in the work instruction information includes at least a text for instructing an action, a figure of an action, a movie of an action, and augmented reality (AR) information of an action.

6. The work support apparatus according to claim 1, wherein
the work instruction information includes an attention item relating to efficiency or quality of a work.

7. The work support apparatus according to claim 1, wherein
when a deviation between the physical characteristic in the sensing information and the physical characteristic in the model is equal to or larger than a predetermined value, the work capacity evaluation processing unit changes the physical characteristic in the model.

8. The work support apparatus according to claim 1, wherein
the work sensing processing unit receives an input of a psychological characteristic of the worker in each work process and stores the psychological characteristic in the sensing information.

9. The work support apparatus according to claim 2, wherein
the work sensing processing unit calculates an index value indicating a predetermined psychological characteristic using the physical characteristic of the worker in each work process, and stores the index value in the sensing information, and
the process allocation processing unit changes an allocation of the work processes performed by the workers, by using the index value of the sensing information included in the work capacity evaluation information and the work process order included in the process information, and outputs process allocation information.

10. The work support apparatus according to claim 1, wherein
a change guideline of the instruction of the work procedure in the work instruction information is associated with the model of the work capacity evaluation information,
the work support apparatus further comprises a work instruction generation processing unit that specifies, for each of the workers, either a granularity or a type, or both a granularity and a type of the instruction of the work procedure in the work instruction information according to the change guideline, and
the work instruction generation processing unit generates the work instruction information when an approval for the work instruction information specified according to the change guideline is obtained from the worker.

11. The work support apparatus according to claim 1, wherein
a change guideline of the instruction of the work procedure in the work instruction information is associated with the model of the work capacity evaluation information,
the work support apparatus further comprises a work instruction generation processing unit that specifies, for each of the workers, either a granularity or a type, or both a granularity and a type of the instruction of the work procedure in the work instruction information according to the change guideline, and
the work instruction generation processing unit generates the work instruction information in which, for each of the workers, either the granularity or the type, or both the granularity and the type are changed in a predetermined cycle according to another change guideline that is different from the change guideline of the worker.

12. The work support apparatus according to claim 1, wherein
a change guideline of the instruction of the work procedure in the work instruction information is associated with the model of the work capacity evaluation information, the work support apparatus further comprises a work instruction generation processing unit that specifies, for each of the workers, either a granularity or a type, or both a granularity and a type of the instruction of the work procedure in the work instruction information according to the change guideline, and
the work instruction generation processing unit stores the work instruction information specified according to the change guideline in the storage unit and performs respective processings of the work sensing processing unit and the work capacity evaluation processing unit again.

13. A work support system using a work support apparatus, wherein
the work support apparatus comprising:
a storage unit that stores work instruction information including instructions of work procedures for instructing workers to perform predetermined work processes and work capacity model information including a plurality of models determined based on physical characteristics of the workers in respective work processes;
a processing unit; and
a communication unit that communicates with a device that controls a production line, and
the processing unit performs:
a work sensing step of sensing the physical characteristic of the worker during a work that the worker performs based on the work instruction information and generating sensing information,
a work capacity evaluation step of selecting, for each of the workers, one or a plurality of models corresponding to the sensing information from the work capacity model information and associating the selected model with the sensing information to generate work capacity evaluation information, and
a step of transmitting work instruction information updated based on the work capacity evaluation information to the device that controls a production line.

14. A work support method using a work support apparatus including:
a storage unit that stores work instruction information including instructions of work procedures for instructing workers to perform predetermined work processes and work capacity model information including a plurality of models determined based on physical characteristics of the workers in respective work processes; and
a processing unit, the work support method comprising:
the processing unit performing:
a work sensing step of sensing the physical characteristic of the worker during a work that the worker performs based on the work instruction information and generating sensing information, and
a work capacity evaluation step of selecting, for each of the workers, one or a plurality of models corresponding to the sensing information from the work capacity model information and associating the selected model with the sensing information to generate work capacity evaluation information.
